(19) United States
(12) Patent Application Publication
(10) Pub. No.: US 2016/0355581 A1
(43) Pub. Date: Dec. 8, 2016

(54) COMPOSITIONS AND METHODS FOR TREATING AMYLOID PLAQUE ASSOCIATED SYMPTOMS

(71) Applicant: The Washington University, St. Louis, MO (US)

(72) Inventors: David Holtzman, St. Louis, MO (US); Jungsu KIM, St. Louis, MO (US); Hong Jiang, St. Louis, MO (US); Adam Eltorai, St. Louis, MO (US)

(21) Appl. No.: 15/243,495

(22) Filed: Aug. 22, 2016

Related U.S. Application Data
(63) Continuation of application No. 13/990,777, filed on Oct. 14, 2013, now abandoned.
(60) Provisional application No. 61/419,060, filed on Dec. 2, 2010.
(60) Provisional application No. 61/548,542, filed on Oct. 18, 2011.

(51) Int. Cl. C07K 16/18

(52) U.S. Cl. CPC C07K 16/18; A61K 2039/54

(57) ABSTRACT

The present invention encompasses compositions and methods for effectively treating at least one symptom or sign of Aβ plaque associated symptoms or for decreasing amyloid plaque loads. The method comprises administering an effective amount of an anti-ApoE antibody to a living mammalian biosystem such as to a human.

### CROSS-REFERENCE TO RELATED APPLICATIONS

This application is a continuation of U.S. Ser. No. 13/990,777, filed Oct. 14, 2013; which is a National Phase filing under 35 U.S.C. §371 of International Application No. PCT/US2011/063121 having an international filing date of Dec. 2, 2011; which claims priority to U.S. Provisional Application Nos. 61/419,060, filed Dec. 2, 2010 and 61/548,542, filed Oct. 18, 2011; the entire content of each of which is incorporated herein by reference.

### GOVERNMENT SUPPORT

This invention was made with government support under AG013956 awarded by the NIH. The government has certain rights in the invention.

### FIELD OF THE INVENTION

The invention relates to compositions and methods for delaying or preventing Aβ plaque associated symptoms, such as those associated with Alzheimer's Disease (AD) in a subject. In particular, the invention relates to modulating the concentration of amyloid-β (Aβ) in the brain of a subject.

### BACKGROUND OF THE INVENTION

Alzheimer's Disease (AD) is the most common cause of dementia and is an increasing public health problem. It is currently estimated to afflict 5 million people in the United States, with an expected increase to 13 million by the year 2050. Alzheimer's Disease leads to loss of memory, cognitive function, and ultimately loss of independence. It takes a heavy personal and financial toll on the subject and the family. Because of the severity and increasing prevalence of the disease in the population, it is urgent that better treatments be developed.

Biochemical, genetic, and animal model evidence implicates amyloid-β (Aβ) as a pathogenic peptide in AD. The neuropathologic and neurochemical hallmarks of AD include synaptic loss and selective neuronal death, a decrease in certain neurotransmitters, and the presence of abnormal proteinaceous deposits within neurons (neurofibrillary tangles) and in the extracellular space (cerebrovascular, diffuse, and neuritic plaques). The main constituent of these plaques is Aβ, a 38-43 amino acid sequence peptide cleaved from the amyloid precursor protein (APP).

Throughout life, soluble Aβ is secreted primarily by neurons, but also other cell types. Excessive Aβ deposition may result from increased Aβ synthesis, as occurs in familial early-onset AD, or decreased Aβ clearance in brain. The lack of compelling evidence that Aβ production occurs in the more common late-onset forms of AD suggests that insufficient Aβ clearance may drive Aβ deposition and amyloid plaque formation as well.

The apolipoprotein E (ApoE) gene remains the most widely replicated genetic risk factor for late-onset AD, with carriers of the E4 allelle having a 3-15-fold greater risk as well as an earlier age of disease onset. In brain, ApoE is mainly synthesized and secreted by astrocytes and microglia which are found to surround amyloid plaques. The present invention provides ApoE antibodies effective at reducing amyloid plaque load in AD mouse models.

### SUMMARY OF THE INVENTION

One aspect of the invention encompasses a method of effectively treating at least one clinically detectable Aβ plaque associated symptom which comprises administering an effective amount of an anti-ApoE antibody to a living human subject. In another aspect, the invention encompasses an antibody useful in such treatment. For instance, an antibody that therapeutically attenuates the toxic effects of the Aβ peptide in a living mammal.

Another aspect of the invention encompasses a composition comprising at least one anti-ApoE antibody. In an aspect, the invention encompasses a composition comprising at least one anti-ApoE antibody produced from hybridoma HJ6.1, HJ6.2, HJ6.3, HJ6.4 or a combination thereof. In an aspect, the invention encompasses a medicinal composition comprising at least one anti-ApoE antibody. In an aspect, the invention encompasses a medicinal composition comprising at least one anti-ApoE antibody produced from hybridoma HJ6.1, HJ6.2, HJ6.3, HJ6.4 or a combination thereof.

Yet another aspect of the invention encompasses a medicinal composition useful to treat at least one clinically detectable Aβ plaque associated symptom. The composition comprises a medicinally effective amount of an anti-ApoE antibody adapted for administration to a living human subject. In an aspect, an antibody useful in such treatment includes an antibody that therapeutically attenuates the toxic effects of the Aβ peptide in a living mammal. In an aspect, the medicinal composition is effectively administered to a living subject.

Still another aspect the invention encompasses a medicinal kit comprising a container containing a functional therapeutic medicinal composition of a medicinally effective amount of an anti-ApoE antibody adapted for administration to a living human subject and any medical devices to be used for said administration. In an aspect, an antibody useful in such treatment includes an antibody that therapeutically attenuates the toxic effects of the Aβ peptide in a living mammal.

Other aspects and iterations of the invention are detailed below.

### BRIEF DESCRIPTION OF THE FIGURES

FIG. 1 graphically depicts a strong effect of anti-ApoE antibodies in decreased amyloid plaque load in samples of hippocampus from PS/APP mice treated intraperitoneally once weekly with 10 mg/kg beginning at 3 months of age until 7 months of age with either an anti-Aβ antibody (HJ3.4) or one of two anti-ApoE antibodies (HJ6.2 or HJ6.3). The control group was treated with phosphate buffered saline (PBS). Animal numbers assessed are N=7 (PBS), N=16 (HJ3.4), N=16 (HJ6.2), N=16).

FIG. 2 graphically depicts a strong effect of anti-ApoE antibodies in decreased amyloid plaque load in samples of brain cortex from PS/APP mice treated intraperitoneally once weekly with 10 mg/kg with either an anti-Aβ antibody (HJ3.4) or one of two anti-ApoE antibodies (HJ6.2 or HJ6.3). The control group was treated with PBS. Animal numbers assessed are N=7 (PBS), N=16 (HJ3.4), N=16 (HJ6.2), N=16).

FIG. 3 graphically depicts binding of anti-apoE antibodies HJ6.1, HJ6.2, HJ6.3 and HJ6.4 to human apoE using an ELISA assay.

FIG. 4 graphically depicts (A) binding of anti-apoE antibodies HJ6.1, HJ6.2, HJ6.3 and HJ6.4 to human apoE in plasma, and (B) levels of cholesterol in the same mice that received the HJ6.1, HJ6.2, HJ6.3 and HJ6.4 antibodies.

FIG. 5 graphically depicts the presence of anti-apoE antibodies HJ6.1, HJ6.2, HJ6.3 and HJ6.4 in the central nervous system after peripheral administration.

FIG. 6 graphically depicts microglial activation after short term administration of HJ6.2 and HJ6.3 (anti-apoE antibodies), and HJ3.4 (an anti-Aβ antibody) to mice over 10 days.

FIG. 7 depicts the amino acid sequences of (A) HJ6.1 heavy chain variable region and first constant region (SEQ ID NO:4); (B) HJ6.1 light chain variable region and constant region (SEQ ID NO:2); (C) HJ6.2 heavy chain variable region and first constant region (SEQ ID NO:8); (D) HJ6.2 light chain variable region and constant region (SEQ ID NO:6); (E) HJ6.3 heavy chain variable region and first constant region (SEQ ID NO:12); (F) HJ6.3 light chain variable region and constant region (SEQ ID NO:10); (G) HJ6.4 heavy chain variable region and first constant region (SEQ ID NO:16); (H) HJ6.4 light chain variable region and constant region (SEQ ID NO:14). Regions comprising CDRs are highlighted.

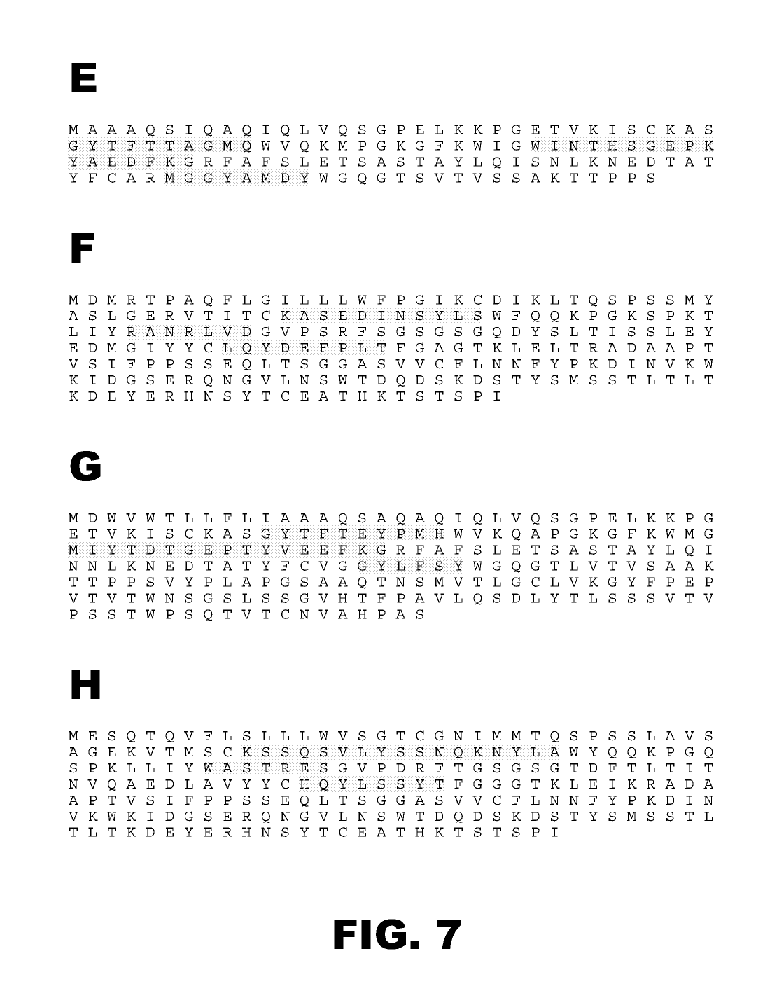

### DETAILED DESCRIPTION

Applicants have discovered antibodies and methods of use thereof for effectively treating Aβ plaque associated symptoms. The method comprises effectively administering a pharmacologically effective amount of anti-ApoE antibody to a living subject. The present invention encompasses the discovery that anti-ApoE antibodies provide a treatment for subjects suffering from Aβ plaque associated symptoms. Thus, the invention provides evidence that signs and symptoms of Aβ plaque associated symptoms may be due, at least in part, to the deleterious effects of ApoE. In an aspect, at least one preclinical or clinical symptom or sign is presented by that subject. In an aspect, an antibody useful in such treating includes an antibody that therapeutically attenuates the toxic effects of the Aβ peptide in a living mammal. In an aspect, antibodies useful in such treating include those which bind an epitope within ApoE.

In an aspect, an anti-ApoE antibody is admixed with at least one suitable compatible adjuvant or excipient resulting in a therapeutic medicinal composition which is capably and effectively administered (given) to a living subject, such as to a human afflicted with Aβ plaque associated symptoms. Typically this is an aqueous composition of high purity.

As used herein, the terms “treating” or “treatment” include prevention, attenuation, reversal, or improvement in at least one symptom or sign of Aβ plaque associated symptoms.

As used herein the term “therapeutically attenuate” includes inducing a change or having a beneficial positive effect resulting therefrom.

One definition of Aβ plaque associated symptoms refers to any symptom caused by the formation of amyloid plaques being composed of regularly ordered fibrillar aggregates called amyloid fibrils. Exemplary disorders that have Aβ plaque associated symptoms include, but are not limited to, Alzheimer's Disease, Lewy body dementia, and cerebral amyloid angiopathy.

Exemplary Aβ plaque associated symptoms may include impaired cognitive function, altered behavior, emotional dysregulation, seizures, impaired nervous system structure or function, and an increased risk of development of Alzheimer's disease. Impaired cognitive function may include but is not limited to difficulties with memory, attention, concentration, language, abstract thought, creativity, executive function, planning, and organization. Altered behavior may include but is not limited to physical or verbal aggression, impulsivity, decreased inhibition, apathy, decreased initiation, changes in personality, abuse of alcohol, tobacco or drugs, and other addiction-related behaviors. Emotional dysregulation may include but is not limited to depression, anxiety, mania, irritability, and emotional incontinence. Seizures may include but are not limited to generalized tonic-clonic seizures, complex partial seizures, and non-epileptic, psychogenic seizures. Impaired nervous system structure or function may include but is not limited to hydrocephalus, Parkinsonism, sleep disorders, psychosis, impairment of balance and coordination. This may include motor impairments such as monoparesis, hemiparesis, tetraparesis, ataxia, ballismus and tremor. This also may include sensory loss or dysfunction including olfactory, tactile, gustatory, visual and auditory sensation. Furthermore, this may include autonomic nervous system impairments such as bowel and bladder dysfunction, sexual dysfunction, blood pressure and temperature dysregulation. Finally, this may include hormonal impairments attributable to dysfunction of the hypothalamus and pituitary gland such as deficiencies and dysregulation of growth hormone, thyroid stimulating hormone, lutenizing hormone, follicle stimulating hormone, gonadotropin releasing hormone, prolactin, and numerous other hormones and modulators. Increased risk of development of Alzheimer's disease includes that risk that is elevated over the expected risk given the subjects age, family history, genetic status and other known risk factors.

Aβ peptides are those derived from a region in the carboxy terminus of a larger protein called amyloid precursor protein (APP). The gene encoding APP is located on chromosome 21. There are many forms of Aβ that may have toxic effects: Aβ peptides are typically 38-43 amino acid sequences long, though they can have truncations and modifications changing their overall size. They can be found in soluble and insoluble compartments, in monomeric, oligomeric and aggregated forms, intracellularly or extracellularly, and may be complexed with other proteins or molecules. The adverse or toxic effects of Aβ may be attributable to any or all of the above noted forms, as well as to others not described specifically.

In one embodiment, the invention provides a method for decreasing the amyloid plaque load in the brain of a subject. The method comprises administering a therapeutically effective amount of an antibody that specifically binds to ApoE to the subject. Suitable antibodies include those disclosed herein. In an exemplary embodiment, a suitable Ab comprises an antibody delineated in Table A below. A method of the invention may decrease the amyloid plaque load in the hippocampus of a subject. A method of the invention may also decrease the amyloid plaque load in the brain cortex of a subject. In each of the above embodiments, the amyloid plaque load may be decreased by at least 1, 2, 3, 4, 5, 6, 7, 8, 9, 10, 11, 12, 13, 14, 15, 16, 17, 18, 19, or 20% compared to untreated or negative control treated subjects. In some embodiments, the amyloid plaque load may be decreased by at least 20, 25, 30, 35, 40, 45, 50, 55, 60, 65, 70, 75, 80, 85, 90, or 95% compared to untreated or negative control treated subjects. In other embodiments, the amyloid plaque load may be decreased by at least 100, 125, 150, 200, 250, 300, 350, 400, or 450% compared to untreated or negative control treated subjects. Methods of measuring amyloid plaque load are known in the art.

Anti-ApoE antibodies useful herein include all antibodies that therapeutically attenuate the adverse or toxic effects of Aft Useful antibodies include but are not limited to those that specifically bind to an epitope within the ApoE coding sequence. Anti-ApoE antibodies useful herein include also antibodies that attenuate the adverse or toxic effects of Aβ and bind to specific regions of ApoE and to other forms of ApoE. Specific regions of ApoE include, but are not limited to, the C-terminal, the N-terminal, and other central domains. Other forms of ApoE include but are not limited to truncated, modified, soluble, insoluble, intracellular, extracellular, monomeric ApoE, oligomeric ApoE, fibrillar, aggregated ApoE or ApoE complexed with other proteins or molecules.

In an aspect, antibodies useful herein include those antibodies which have been isolated, characterized, purified, are functional and have been recovered (obtained) for use in a functional therapeutic composition which is administered to a living subject having Aβ plaque associated symptoms.

“Monoclonal antibody” refers to an antibody that is derived from a single copy or clone, including e.g., any eukaryotic, prokaryotic, or phage clone. “Monoclonal antibody” is not limited to antibodies produced through hybridoma technology. Monoclonal antibodies can be produced using e.g., hybridoma techniques well known in the art, as well as recombinant technologies, phage display technologies, synthetic technologies or combinations of such technologies and other technologies readily known in the art. Furthermore, the monoclonal antibody may be labeled with a detectable label, immobilized on a solid phase and/or conjugated with a heterologous compound (e.g., an enzyme or toxin) according to methods known in the art.

Further by “antibody” is meant a functional monoclonal antibody, or an immunologically effective fragment thereof; such as an Fab, Fab′, or F(ab′)2 fragment thereof. In some contexts herein, fragments will be mentioned specifically for emphasis; nevertheless, it will be understood that regardless of whether fragments are specified, the term “antibody” includes such fragments as well as single-chain forms. As long as the protein retains the ability specifically to bind its intended target, it is included within the term “antibody.” Also included within the definition “antibody” for example are single chain forms, generally designated Fv, regions, of antibodies with this specificity. Preferably, but not necessarily, the antibodies useful in the discovery are produced recombinantly, as manipulation of the typically murine or other non-human antibodies with the appropriate specificity is required in order to convert them to humanized form. Antibodies may or may not be glycosylated, though glycosylated antibodies are preferred. Antibodies are properly cross-linked via disulfide bonds, as is known.

The basic antibody structural unit of an antibody useful herein comprises a tetramer. Each tetramer is composed of two identical pairs of polypeptide chains, each pair having one “light’ (about 25 kDa) and one “heavy” chain (about 50-70 kDa). The amino-terminal portion of each chain includes a variable region of about 100 to 110 or more amino acid sequences primarily responsible for antigen recognition. The carboxy-terminal portion of each chain defines a constant region primarily responsible for effector function.

Anti-ApoE antibodies useful herein include those which are isolated, characterized, purified, functional and have been recovered (obtained) from a process for their preparation and thus available for use herein in a useful form in a therapeutically and medicinally sufficient amount.

Light chains are classified as gamma, mu, alpha, and lambda. Heavy chains are classified as gamma, mu, alpha, delta, or epsilon, and define the antibody's isotype as IgO, IgM, IgA, IgD and IgE, respectively. Within light and heavy chains, the variable and constant regions are joined by a “J” region of about 12 or more amino acid sequences, with the heavy chain also including a “D” region of about 10 more amino acid sequences.

The variable regions of each light/heavy chain pair form the antibody binding site. Thus, an intact antibody has two binding sites. The chains exhibit the same general structure of relatively conserved framework regions (FR) joined by three hypervariable regions, also called complementarily determining regions (hereinafter referred to as “CDRs.”) The CDRs from the two chains are aligned by the framework regions, enabling binding to a specific epitope. From N-terminal to C-terminal, both light and heavy chains comprise the domains FR1, CDR1, FR2, CDR2, FR3, CDR3 and FR4 respectively. The assignment of amino acid sequences to each domain is in accordance with known conventions (See, Kabat “Sequences of Proteins of Immunological Interest” National Institutes of Health, Bethesda, Md., 1987 and 1991; Chothia, et al, J. Mol. Bio. (1987) 196:901-917; Chothia, et al., Nature (1989) 342:878-883).

In an aspect, monoclonal anti-apoE antibodies are generated with appropriate specificity by standard techniques of immunization of mammals, forming hybridomas from the antibody-producing cells of said mammals or otherwise immortalizing them, and culturing the hybridomas or immortalized cells to assess them for the appropriate specificity. In the present case, such antibodies could be generated by immunizing a human, rabbit, rat or mouse, for example, with a peptide representing an epitope encompassing a region of the ApoE protein coding sequence or an appropriate subregion thereof. Materials for recombinant manipulation can be obtained by retrieving the nucleotide sequences encoding the desired antibody from the hybridoma or other cell that produces it. These nucleotide sequences can then be manipulated and isolated, characterized, purified and, recovered to provide them in humanized form, for use herein if desired.

As used herein “humanized antibody” includes an anti-ApoE antibody that is composed partially or fully of amino acid sequence sequences derived from a human antibody germline by altering the sequence of an antibody having non-human complementarity determining regions (“CDR”). The simplest such alteration may consist simply of substituting the constant region of a human antibody for the murine constant region, thus resulting in a human/murine chimera which may have sufficiently low immunogenicity to be acceptable for pharmaceutical use. Preferably, however, the variable region of the antibody and even the CDR is also humanized by techniques that are by now well known in the art. The framework regions of the variable regions are substituted by the corresponding human framework regions leaving the non-human CDR substantially intact, or even replacing the CDR with sequences derived from a human genome. CDRs may also be randomly mutated such that binding activity and affinity for ApoE is maintained or enhanced in the context of fully human germline framework regions or framework regions that are substantially human. Substantially human frameworks have at least 90%, 95%, or 99% sequence identity with a known human framework sequence. Fully useful human antibodies are produced in genetically modified mice whose immune systems have been altered to correspond to human immune systems. As mentioned above, it is sufficient for use in the methods of this discovery, to employ an immunologically specific fragment of the antibody, including fragments representing single chain forms.

Further, as used herein the term “humanized antibody” refers to an anti-ApoE antibody comprising a human framework, at least one CDR from a nonhuman antibody, and in which any constant region present is substantially identical to a human immunoglobulin constant region, i.e., at least about 85-90%, preferably at least 95% identical. Hence, all parts of a humanized antibody, except possibly the CDRs, are substantially identical to corresponding pairs of one or more native human immunoglobulin sequences.

If desired, the design of humanized immunoglobulins may be carried out as follows. When an amino acid sequence falls under the following category, the framework amino acid sequence of a human immunoglobulin to be used (acceptor immunoglobulin) is replaced by a framework amino acid sequence from a CDR-providing nonhuman immunoglobulin (donor immunoglobulin): (a) the amino acid sequence in the human framework region of the acceptor immunoglobulin is unusual for human immunoglobulin at that position, whereas the corresponding amino acid sequence in the donor immunoglobulin is typical for human immunoglobulin at that position; (b) the position of the amino acid sequence is immediately adjacent to one of the CDRs; or (c) any side chain atom of a framework amino acid sequence is within about 5-6 angstroms (center-to-center) of any atom of a CDR amino acid sequence in a three dimensional immunoglobulin model (Queen, et al., op. cit., and Co, et al, Proc. Natl. Acad. Sci. USA (1991) 88:2869). When each of the amino acid sequences in the human framework region of the acceptor immunoglobulin and a corresponding amino acid sequence in the donor immunoglobulin is unusual for human immunoglobulin at that position, such an amino acid sequence is replaced by an amino acid sequence typical for human immunoglobulin at that position.

In all instances, an antibody of the invention specifically binds ApoE. In exemplary embodiments, an antibody of the invention specifically binds human ApoE. The phrase “specifically binds” herein means antibodies bind to the protein with a binding constant in the range of at least 10⁻⁴-10⁻⁶ M⁻¹, with a preferred range being 10⁻⁷-10⁻⁹ M⁻¹. The sequence of ApoE from a variety of species is known in the art, and methods of determining whether an antibody binds to ApoE are known in the art. For instance, see the Examples. An antibody of the invention may recognize ApoE2, ApoE3, ApoE4, or an allelic variant thereof. In one embodiment, an antibody of the invention may recognize human ApoE4.

A preferred antibody is a humanized form of mouse antibody derived from a hybridoma designated HJ6.1 (ATCC Patent Deposit Designation PT-11805), HJ6.2 (ATCC Patent Deposit Designation PT-11806), HJ6.3 (ATCC Patent Deposit Designation PT-11807), or HJ6.4 (ATCC Patent Deposit Designation PT-11808). As used herein, the term “derived from” means that the “derived” antibody comprises at least one CDR region from the antibody produced by HJ6.1, HJ6.2, HJ6.3, or HJ6.4. Stated another way, the “derived antibody” comprises at least one CDR region comprised of the amino acid sequence selected from the group consisting of SEQ ID NO: 17, 18, 19, 20, 21, 22, 23, 24, 25, 26, 27, 28, 29, 30, 31, 32, 33, 34, 35, 36, 37, 38, and 39.

In one embodiment, an antibody of the invention may be derived from the hybridoma HJ6.1, and may be encoded by a nucleic acid sequence comprising 90, 91, 92, 93, 94, 95, 96, 97, 98, or 99% identity to the light chain variable region of SEQ ID NO:1, or may be encoded by a nucleic acid sequence comprising 90, 91, 92, 93, 94, 95, 96, 97, 98, or 99% identity to the heavy chain variable region of SEQ ID NO:2. In another embodiment, an antibody of the invention may be derived from the hybridoma HJ6.1, and may comprise an amino acid sequence with 90, 91, 92, 93, 94, 95, 96, 97, 98, or 99% identity to the light chain variable region of SEQ ID NO:3, or may comprise an amino acid sequence with 90, 91, 92, 93, 94, 95, 96, 97, 98, or 99% identity to the heavy chain variable region of SEQ ID NO:4. In each of the above embodiments, the antibody may be humanized.

In yet another embodiment, an antibody of the invention may be derived from the hybridoma HJ6.2, and may be encoded by a nucleic acid sequence comprising 90, 91, 92, 93, 94, 95, 96, 97, 98, or 99% identity to the light chain variable region of SEQ ID NO:5, or may be encoded by a nucleic acid sequence comprising 90, 91, 92, 93, 94, 95, 96, 97, 98, or 99% identity to the heavy chain variable region of SEQ ID NO:6. In another embodiment, an antibody of the invention may be derived from the hybridoma HJ6.2, and may comprise an amino acid sequence with 90, 91, 92, 93, 94, 95, 96, 97, 98, or 99% identity to the light chain variable region of SEQ ID NO:7, or may comprise an amino acid sequence with 90, 91, 92, 93, 94, 95, 96, 97, 98, or 99% identity to the heavy chain variable region of SEQ ID NO:8. In each of the above embodiments, the antibody may be humanized.

In an additional embodiment, an antibody of the invention may be derived from the hybridoma HJ6.3, and may be encoded by a nucleic acid sequence comprising 90, 91, 92, 93, 94, 95, 96, 97, 98, or 99% identity to the light chain variable region of SEQ ID NO:9, or may be encoded by a nucleic acid sequence comprising 90, 91, 92, 93, 94, 95, 96, 97, 98, or 99% identity to the heavy chain variable region of SEQ ID NO:10. In another embodiment, an antibody of the invention may be derived from the hybridoma HJ6.3, and may comprise an amino acid sequence with 90, 91, 92, 93, 94, 95, 96, 97, 98, or 99% identity to the light chain variable region of SEQ ID NO:11, or comprise an amino acid sequence with 90, 91, 92, 93, 94, 95, 96, 97, 98, or 99% identity to the heavy chain variable region of SEQ ID NO:12. In each of the above embodiments, the antibody may be humanized.

In still another embodiment, an antibody of the invention may be derived from the hybridoma HJ6.4, and may have a nucleic acid sequence with 90, 91, 92, 93, 94, 95, 96, 97, 98, or 99% identity to the light chain variable region of SEQ ID NO:13, and a nucleic acid sequence with 90, 91, 92, 93, 94, 95, 96, 97, 98, or 99% identity to the heavy chain variable region of SEQ ID NO:14. In another embodiment, an antibody of the invention may be derived from the hybridoma HJ6.4, and may have an amino acid sequence with 90, 91, 92, 93, 94, 95, 96, 97, 98, or 99% identity to the light chain variable region of SEQ ID NO:15, or an amino acid sequence with 90, 91, 92, 93, 94, 95, 96, 97, 98, or 99% identity to the heavy chain variable region of SEQ ID NO:16. In each of the above embodiments, the antibody may be humanized.

In an exemplary embodiment of an antibody of the invention that binds to anti-apoE, the antibody comprises the light chain nucleic acid sequence of SEQ ID NO:1 and the heavy chain nucleic acid sequence of SEQ ID NO:2 [i.e. the monoclonal antibody referred to herein as HJ6.1]. In another exemplary embodiment of an antibody of the invention that binds to anti-apoE, the antibody comprises the light chain amino acid sequence of SEQ ID NO:3 and the heavy chain amino acid sequence of SEQ ID NO:4 [i.e. the monoclonal antibody referred to herein as HJ6.1]. In yet another exemplary embodiment of an antibody of the invention that binds to anti-apoE, the antibody comprises the light chain nucleic acid sequence of SEQ ID NO:5 and the heavy chain nucleic acid sequence of SEQ ID NO:6 [i.e. the monoclonal antibody referred to herein as HJ6.2]. In still another exemplary embodiment of an antibody of the invention that binds to anti-apoE, the antibody comprises the light chain amino acid sequence of SEQ ID NO:7 and the heavy chain amino acid sequence of SEQ ID NO:8 [i.e. the monoclonal antibody referred to herein as HJ6.2].

In certain embodiments, an antibody of the invention is encoded by the light chain nucleic acid sequence of SEQ ID NO:9 and the heavy chain nucleic acid sequence of SEQ ID NO:10 [i.e. the monoclonal antibody referred to herein as HJ6.3]. In other embodiments, an antibody of the invention is encoded by the light chain amino acid sequence of SEQ ID NO:11 and the heavy chain amino acid sequence of SEQ ID NO:12 [i.e. the monoclonal antibody referred to herein as HJ6.3]. In another embodiment, an antibody of the invention is encoded by the light chain nucleic acid sequence of SEQ ID NO:13 and the heavy chain nucleic acid sequence of SEQ ID NO:14 [i.e. the monoclonal antibody referred to herein as HJ6.4]. In yet another embodiment, an antibody of the invention is encoded by the light chain amino acid sequence of SEQ ID NO:15 and the heavy chain amino acid sequence of SEQ ID NO:16 [i.e. the monoclonal antibody referred to herein as HJ6.4].

In one embodiment, an antibody of the invention may comprise a light chain CDR1, such as the antibodies 1, 49, 97, and 145 of Table A. In another embodiment, an antibody of the invention may comprise a light chain CDR2, such as the antibodies 4, 52, 100, and 148 of Table A. In yet another embodiment, an antibody of the invention may comprise a light chain CDR3, such as the antibodies 6, 54, 102, and 150 of Table A. In an alternative embodiment, an antibody of the invention may comprise a combination of two or three light chain CDRs, such as the antibodies 2, 3, 5, 50, 51, 53, 98, 99, 101, 146, 147, and 149 of Table A.

Similarly, in one embodiment, an antibody of the invention may comprise a heavy chain CDR1, such as the antibodies 7, 55, 103, and 151 of Table A. In another embodiment, an antibody of the invention may comprise a heavy chain CDR2, such as the antibodies 10, 58, 106, and 154 of Table A. In yet another embodiment, an antibody of the invention may comprise a heavy chain CDR3, such as the antibodies 12, 60, 108, and 156 of Table A. In an alternative embodiment, an antibody of the invention may comprise a combination of two or three heavy chain CDRs, such as the antibodies 8, 9, 11, 56, 57, 59, 104, 105, 107, 152, 153, and 155 of Table A.

Alternatively, an antibody of the invention may comprise one or more light chain CDRs and one or more heavy chain CDRs, such as the antibodies 13-48, 61-96, 109-144, and 157-192 of Table A.

TABLE A

| Anti- | Light Chain | | | Heavy Chain | | |
|---|---|---|---|---|---|---|
| body | CDR1 | CDR2 | CDR3 | CDR1 | CDR2 | CDR3 |
| 1 | SEQ ID NO:17 | | | | | |
| 2 | SEQ ID NO:17 | SEQ ID NO:18 | | | | |
| 3 | SEQ ID NO:17 | SEQ ID NO:18 | LSP | | | |
| 4 | | SEQ ID NO:18 | | | | |
| 5 | | SEQ ID NO:18 | LSP | | | |
| 6 | | | LSP | | | |
| 7 | | | | SEQ ID NO:19 | | |
| 8 | | | | SEQ ID NO:19 | SEQ ID NO:20 | |
| 9 | | | | SEQ ID NO:19 | SEQ ID NO:20 | SEQ ID NO:21 |
| 10 | | | | | SEQ ID NO:20 | |
| 11 | | | | | SEQ ID NO:20 | SEQ ID NO:21 |
| 12 | | | | | | SEQ ID NO:21 |
| 13 | SEQ ID NO:17 | | | SEQ ID NO:19 | | |
| 14 | SEQ ID NO:17 | | | SEQ ID NO:19 | SEQ ID NO:20 | |
| 15 | SEQ ID NO:17 | | | SEQ ID NO:19 | SEQ ID NO:20 | SEQ ID NO:21 |
| 16 | SEQ ID NO:17 | | | | SEQ ID NO:20 | |
| 17 | SEQ ID NO:17 | | | | SEQ ID NO:20 | SEQ ID NO:21 |
| 18 | SEQ ID NO:17 | | | | | SEQ ID NO:21 |
| 19 | SEQ ID NO:17 | SEQ ID NO:18 | | SEQ ID NO:19 | | |
| 20 | SEQ ID NO:17 | SEQ ID NO:18 | | SEQ ID NO:19 | SEQ ID NO:20 | |
| 21 | SEQ ID NO:17 | SEQ ID NO:18 | | SEQ ID NO:19 | SEQ ID NO:20 | SEQ ID NO:21 |
| 22 | SEQ ID NO:17 | SEQ ID NO:18 | | | SEQ ID NO:20 | |
| 23 | SEQ ID NO:17 | SEQ ID NO:18 | | | SEQ ID NO:20 | SEQ ID NO:21 |
| 24 | SEQ ID NO:17 | SEQ ID NO:18 | | | | SEQ ID NO:21 |
| 25 | SEQ ID NO:17 | SEQ ID NO:18 | LSP | SEQ ID NO:19 | | |
| 26 | SEQ ID NO:17 | SEQ ID NO:18 | LSP | SEQ ID NO:19 | SEQ ID NO:20 | |
| 27 | SEQ ID NO:17 | SEQ ID NO:18 | LSP | SEQ ID NO:19 | SEQ ID NO:20 | SEQ ID NO:21 |
| 28 | SEQ ID NO:17 | SEQ ID NO:18 | LSP | | SEQ ID NO:20 | |
| 29 | SEQ ID NO:17 | SEQ ID NO:18 | LSP | | SEQ ID NO:20 | SEQ ID NO:21 |
| 30 | SEQ ID NO:17 | SEQ ID NO:18 | LSP | | | SEQ ID NO:21 |
| 31 | | SEQ ID NO:18 | | SEQ ID NO:19 | | |
| 32 | | SEQ ID NO:18 | | SEQ ID NO:19 | SEQ ID NO:20 | |
| 33 | | SEQ ID NO:18 | | SEQ ID NO:19 | SEQ ID NO:20 | SEQ ID NO:21 |
| 34 | | SEQ ID NO:18 | | | SEQ ID NO:20 | |
| 35 | | SEQ ID NO:18 | | | SEQ ID NO:20 | SEQ ID NO:21 |
| 36 | | SEQ ID NO:18 | | | | SEQ ID NO:21 |
| 37 | | SEQ ID NO:18 | LSP | SEQ ID NO:19 | | |
| 38 | | SEQ ID NO:18 | LSP | SEQ ID NO:19 | SEQ ID NO:20 | |
| 39 | | SEQ ID NO:18 | LSP | SEQ ID NO:19 | SEQ ID NO:20 | SEQ ID NO:21 |
| 40 | | SEQ ID NO:18 | LSP | | SEQ ID NO:20 | |
| 41 | | SEQ ID NO:18 | LSP | | SEQ ID NO:20 | SEQ ID NO:21 |
| 42 | | SEQ ID NO:18 | LSP | | | SEQ ID NO:21 |
| 43 | | | LSP | SEQ ID NO:19 | | |
| 44 | | | LSP | SEQ ID NO:19 | SEQ ID NO:20 | |
| 45 | | | LSP | SEQ ID NO:19 | SEQ ID NO:20 | SEQ ID NO:21 |
| 46 | | | LSP | | SEQ ID NO:20 | |
| 47 | | | LSP | | SEQ ID NO:20 | SEQ ID NO:21 |
| 48 | | | LSP | | | SEQ ID NO:21 |
| 49 | SEQ ID NO:22 | | | | | |
| 50 | SEQ ID NO:22 | SEQ ID NO:23 | | | | |
| 51 | SEQ ID NO:22 | SEQ ID NO:23 | SEQ ID NO:24 | | | |
| 52 | | SEQ ID NO:23 | | | | |
| 53 | | SEQ ID NO:23 | SEQ ID NO:24 | | | |
| 54 | | | SEQ ID NO:24 | | | |
| 55 | | | | SEQ ID NO:25 | | |
| 56 | | | | SEQ ID NO:25 | SEQ ID NO:26 | |
| 57 | | | | SEQ ID NO:25 | SEQ ID NO:26 | SEQ ID NO:27 |
| 58 | | | | | SEQ ID NO:26 | |
| 59 | | | | | SEQ ID NO:26 | SEQ ID NO:27 |
| 60 | | | | | | SEQ ID NO:27 |
| 61 | SEQ ID NO:22 | | | SEQ ID NO:25 | | |
| 62 | SEQ ID NO:22 | | | SEQ ID NO:25 | SEQ ID NO:26 | |
| 63 | SEQ ID NO:22 | | | SEQ ID NO:25 | SEQ ID NO:26 | SEQ ID NO:27 |
| 64 | SEQ ID NO:22 | | | | SEQ ID NO:26 | |
| 65 | SEQ ID NO:22 | | | | SEQ ID NO:26 | SEQ ID NO:27 |
| 66 | SEQ ID NO:22 | | | | | SEQ ID NO:27 |
| 67 | SEQ ID NO:22 | SEQ ID NO:23 | | SEQ ID NO:25 | | |
| 68 | SEQ ID NO:22 | SEQ ID NO:23 | | SEQ ID NO:25 | SEQ ID NO:26 | |
| 69 | SEQ ID NO:22 | SEQ ID NO:23 | | SEQ ID NO:25 | SEQ ID NO:26 | SEQ ID NO:27 |
| 70 | SEQ ID NO:22 | SEQ ID NO:23 | | | SEQ ID NO:26 | |
| 71 | SEQ ID NO:22 | SEQ ID NO:23 | | | SEQ ID NO:26 | SEQ ID NO:27 |
| 72 | SEQ ID NO:22 | SEQ ID NO:23 | | | | SEQ ID NO:27 |
| 73 | SEQ ID NO:22 | SEQ ID NO:23 | SEQ ID NO:24 | SEQ ID NO:25 | | |
| 74 | SEQ ID NO:22 | SEQ ID NO:23 | SEQ ID NO:24 | SEQ ID NO:25 | SEQ ID NO:26 | |
| 75 | SEQ ID NO:22 | SEQ ID NO:23 | SEQ ID NO:24 | SEQ ID NO:25 | SEQ ID NO:26 | SEQ ID NO:27 |
| 76 | SEQ ID NO:22 | SEQ ID NO:23 | SEQ ID NO:24 | | SEQ ID NO:26 | |
| 77 | SEQ ID NO:22 | SEQ ID NO:23 | SEQ ID NO:24 | | SEQ ID NO:26 | SEQ ID NO:27 |
| 78 | SEQ ID NO:22 | SEQ ID NO:23 | SEQ ID NO:24 | | | SEQ ID NO:27 |
| 79 | | SEQ ID NO:23 | | SEQ ID NO:25 | | |
| 80 | | SEQ ID NO:23 | | SEQ ID NO:25 | SEQ ID NO:26 | |
| 81 | | SEQ ID NO:23 | | SEQ ID NO:25 | SEQ ID NO:26 | SEQ ID NO:27 |
| 82 | | SEQ ID NO:23 | | | SEQ ID NO:26 | |
| 83 | | SEQ ID NO:23 | | | SEQ ID NO:26 | SEQ ID NO:27 |
| 84 | | SEQ ID NO:23 | | | | SEQ ID NO:27 |
| 85 | | SEQ ID NO:23 | SEQ ID NO:24 | SEQ ID NO:25 | | |
| 86 | | SEQ ID NO:23 | SEQ ID NO:24 | SEQ ID NO:25 | SEQ ID NO:26 | |
| 87 | | SEQ ID NO:23 | SEQ ID NO:24 | SEQ ID NO:25 | SEQ ID NO:26 | SEQ ID NO:27 |
| 88 | | SEQ ID NO:23 | SEQ ID NO:24 | | SEQ ID NO:26 | |
| 89 | | SEQ ID NO:23 | SEQ ID NO:24 | | SEQ ID NO:26 | SEQ ID NO:27 |
| 90 | | SEQ ID NO:23 | SEQ ID NO:24 | | | SEQ ID NO:27 |
| 91 | | | SEQ ID NO:24 | SEQ ID NO:25 | | |
| 92 | | | SEQ ID NO:24 | SEQ ID NO:25 | SEQ ID NO:26 | |
| 93 | | | SEQ ID NO:24 | SEQ ID NO:25 | SEQ ID NO:26 | SEQ ID NO:27 |
| 94 | | | SEQ ID NO:24 | | SEQ ID NO:26 | |
| 95 | | | SEQ ID NO:24 | | SEQ ID NO:26 | SEQ ID NO:27 |
| 96 | | | SEQ ID NO:24 | | | SEQ ID NO:27 |
| 97 | SEQ ID NO:28 | | | | | |
| 98 | SEQ ID NO:28 | SEQ ID NO:29 | | | | |
| 99 | SEQ ID NO:28 | SEQ ID NO:29 | SEQ ID NO:30 | | | |
| 100 | | SEQ ID NO:29 | | | | |
| 101 | | SEQ ID NO:29 | SEQ ID NO:30 | | | |
| 102 | | | SEQ ID NO:30 | | | |
| 103 | | | | SEQ ID NO:31 | | |
| 104 | | | | SEQ ID NO:31 | SEQ ID NO:32 | |
| 105 | | | | SEQ ID NO:31 | SEQ ID NO:32 | SEQ ID NO:33 |
| 106 | | | | | SEQ ID NO:32 | |
| 107 | | | | | SEQ ID NO:32 | SEQ ID NO:33 |
| 108 | | | | | | SEQ ID NO:33 |
| 109 | SEQ ID NO:28 | | | SEQ ID NO:31 | | |
| 110 | SEQ ID NO:28 | | | SEQ ID NO:31 | SEQ ID NO:32 | |
| 111 | SEQ ID NO:28 | | | SEQ ID NO:31 | SEQ ID NO:32 | SEQ ID NO:33 |
| 112 | SEQ ID NO:28 | | | | SEQ ID NO:32 | |
| 113 | SEQ ID NO:28 | | | | SEQ ID NO:32 | SEQ ID NO:33 |
| 114 | SEQ ID NO:28 | | | | | SEQ ID NO:33 |
| 115 | SEQ ID NO:28 | SEQ ID NO:29 | | SEQ ID NO:31 | | |
| 116 | SEQ ID NO:28 | SEQ ID NO:29 | | SEQ ID NO:31 | SEQ ID NO:32 | |
| 117 | SEQ ID NO:28 | SEQ ID NO:29 | | SEQ ID NO:31 | SEQ ID NO:32 | SEQ ID NO:33 |
| 118 | SEQ ID NO:28 | SEQ ID NO:29 | | | SEQ ID NO:32 | |
| 119 | SEQ ID NO:28 | SEQ ID NO:29 | | | SEQ ID NO:32 | SEQ ID NO:33 |
| 120 | SEQ ID NO:28 | SEQ ID NO:29 | | | | SEQ ID NO:33 |
| 121 | SEQ ID NO:28 | SEQ ID NO:29 | SEQ ID NO:30 | SEQ ID NO:31 | | |
| 122 | SEQ ID NO:28 | SEQ ID NO:29 | SEQ ID NO:30 | SEQ ID NO:31 | SEQ ID NO:32 | |
| 123 | SEQ ID NO:28 | SEQ ID NO:29 | SEQ ID NO:30 | SEQ ID NO:31 | SEQ ID NO:32 | SEQ ID NO:33 |
| 124 | SEQ ID NO:28 | SEQ ID NO:29 | SEQ ID NO:30 | | SEQ ID NO:32 | |
| 125 | SEQ ID NO:28 | SEQ ID NO:29 | SEQ ID NO:30 | | SEQ ID NO:32 | SEQ ID NO:33 |
| 126 | SEQ ID NO:28 | SEQ ID NO:29 | SEQ ID NO:30 | | | SEQ ID NO:33 |
| 127 | | SEQ ID NO:29 | | SEQ ID NO:31 | | |
| 128 | | SEQ ID NO:29 | | SEQ ID NO:31 | SEQ ID NO:32 | |
| 129 | | SEQ ID NO:29 | | SEQ ID NO:31 | SEQ ID NO:32 | SEQ ID NO:33 |
| 130 | | SEQ ID NO:29 | | | SEQ ID NO:32 | |
| 131 | | SEQ ID NO:29 | | | SEQ ID NO:32 | SEQ ID NO:33 |
| 132 | | SEQ ID NO:29 | | | | SEQ ID NO:33 |
| 133 | | SEQ ID NO:29 | SEQ ID NO:30 | SEQ ID NO:31 | | |
| 134 | | SEQ ID NO:29 | SEQ ID NO:30 | SEQ ID NO:31 | SEQ ID NO:32 | |
| 135 | | SEQ ID NO:29 | SEQ ID NO:30 | SEQ ID NO:31 | SEQ ID NO:32 | SEQ ID NO:33 |
| 136 | | SEQ ID NO:29 | SEQ ID NO:30 | | SEQ ID NO:32 | |
| 137 | | SEQ ID NO:29 | SEQ ID NO:30 | | SEQ ID NO:32 | SEQ ID NO:33 |
| 138 | | SEQ ID NO:29 | SEQ ID NO:30 | | | SEQ ID NO:33 |
| 139 | | | SEQ ID NO:30 | SEQ ID NO:31 | | |
| 140 | | | SEQ ID NO:30 | SEQ ID NO:31 | SEQ ID NO:32 | |
| 141 | | | SEQ ID NO:30 | SEQ ID NO:31 | SEQ ID NO:32 | SEQ ID NO:33 |
| 142 | | | SEQ ID NO:30 | | SEQ ID NO:32 | |
| 143 | | | SEQ ID NO:30 | | SEQ ID NO:32 | SEQ ID NO:33 |
| 144 | | | SEQ ID NO:30 | | | SEQ ID NO:33 |
| 145 | SEQ ID NO:34 | | | | | |
| 146 | SEQ ID NO:34 | SEQ ID NO:35 | | | | |
| 147 | SEQ ID NO:34 | SEQ ID NO:35 | SEQ ID NO:36 | | | |
| 148 | | SEQ ID NO:35 | | | | |
| 149 | | SEQ ID NO:35 | SEQ ID NO:36 | | | |
| 150 | | | SEQ ID NO:36 | | | |
| 151 | | | | SEQ ID NO:37 | | |
| 152 | | | | SEQ ID NO:37 | SEQ ID NO:38 | |
| 153 | | | | SEQ ID NO:37 | SEQ ID NO:38 | SEQ ID NO:39 |
| 154 | | | | | SEQ ID NO:38 | |
| 155 | | | | | SEQ ID NO:38 | SEQ ID NO:39 |
| 156 | | | | | | SEQ ID NO:39 |
| 157 | SEQ ID NO:34 | | | SEQ ID NO:37 | | |
| 158 | SEQ ID NO:34 | | | SEQ ID NO:37 | SEQ ID NO:38 | |
| 159 | SEQ ID NO:34 | | | SEQ ID NO:37 | SEQ ID NO:38 | SEQ ID NO:39 |
| 160 | SEQ ID NO:34 | | | | SEQ ID NO:38 | |
| 161 | SEQ ID NO:34 | | | | SEQ ID NO:38 | SEQ ID NO:39 |
| 162 | SEQ ID NO:34 | | | | | SEQ ID NO:39 |
| 163 | SEQ ID NO:34 | SEQ ID NO:35 | | SEQ ID NO:37 | | |
| 164 | SEQ ID NO:34 | SEQ ID NO:35 | | SEQ ID NO:37 | SEQ ID NO:38 | |
| 165 | SEQ ID NO:34 | SEQ ID NO:35 | | SEQ ID NO:37 | SEQ ID NO:38 | SEQ ID NO:39 |
| 166 | SEQ ID NO:34 | SEQ ID NO:35 | | | SEQ ID NO:38 | |
| 167 | SEQ ID NO:34 | SEQ ID NO:35 | | | SEQ ID NO:38 | SEQ ID NO:39 |
| 168 | SEQ ID NO:34 | SEQ ID NO:35 | | | | SEQ ID NO:39 |
| 169 | SEQ ID NO:34 | SEQ ID NO:35 | SEQ ID NO:36 | SEQ ID NO:37 | | |
| 170 | SEQ ID NO:34 | SEQ ID NO:35 | SEQ ID NO:36 | SEQ ID NO:37 | SEQ ID NO:38 | |
| 171 | SEQ ID NO:34 | SEQ ID NO:35 | SEQ ID NO:36 | SEQ ID NO:37 | SEQ ID NO:38 | SEQ ID NO:39 |
| 172 | SEQ ID NO:34 | SEQ ID NO:35 | SEQ ID NO:36 | | SEQ ID NO:38 | |
| 173 | SEQ ID NO:34 | SEQ ID NO:35 | SEQ ID NO:36 | | SEQ ID NO:38 | SEQ ID NO:39 |
| 174 | SEQ ID NO:34 | SEQ ID NO:35 | SEQ ID NO:36 | | | SEQ ID NO:39 |
| 175 | | SEQ ID NO:35 | | SEQ ID NO:37 | | |
| 176 | | SEQ ID NO:35 | | SEQ ID NO:37 | SEQ ID NO:38 | |
| 177 | | SEQ ID NO:35 | | SEQ ID NO:37 | SEQ ID NO:38 | SEQ ID NO:39 |
| 178 | | SEQ ID NO:35 | | | SEQ ID NO:38 | |
| 179 | | SEQ ID NO:35 | | | SEQ ID NO:38 | SEQ ID NO:39 |
| 180 | | SEQ ID NO:35 | | | | SEQ ID NO:39 |
| 181 | | SEQ ID NO:35 | SEQ ID NO:36 | SEQ ID NO:37 | | |
| 182 | | SEQ ID NO:35 | SEQ ID NO:36 | SEQ ID NO:37 | SEQ ID NO:38 | |
| 183 | | SEQ ID NO:35 | SEQ ID NO:36 | SEQ ID NO:37 | SEQ ID NO:38 | SEQ ID NO:39 |
| 184 | | SEQ ID NO:35 | SEQ ID NO:36 | | SEQ ID NO:38 | |
| 185 | | SEQ ID NO:35 | SEQ ID NO:36 | | SEQ ID NO:38 | SEQ ID NO:39 |
| 186 | | SEQ ID NO:35 | SEQ ID NO:36 | | | SEQ ID NO:39 |
| 187 | | | SEQ ID NO:36 | SEQ ID NO:37 | | |
| 188 | | | SEQ ID NO:36 | SEQ ID NO:37 | SEQ ID NO:38 | |
| 189 | | | SEQ ID NO:36 | SEQ ID NO:37 | SEQ ID NO:38 | SEQ ID NO:39 |
| 190 | | | SEQ ID NO:36 | | SEQ ID NO:38 | |
| 191 | | | SEQ ID NO:36 | | SEQ ID NO:38 | SEQ ID NO:39 |
| 192 | | | SEQ ID NO:36 | | | SEQ ID NO:39 |

In various embodiments, an antibody of the invention is humanized. For instance, in one embodiment, a humanized antibody of the invention may comprise a light chain variable region comprising a CDR1 of amino acid sequence SEQ ID NO: 17 with zero to two amino acid substitutions, a CDR2 of amino acid sequence SEQ ID NO: 18 with zero to two amino acid substitutions, and a CDR3 of amino acid sequence LSP, or may comprise a heavy chain variable region comprising a CDR1 of amino acid sequence SEQ ID NO: 19 with zero to two amino acid substitutions, a CDR2 of amino acid sequence SEQ ID NO: 20 with zero to two amino acid substitutions, and a CDR3 of amino acid sequence SEQ ID NO: 21 with zero to two amino acid substitutions. In a preferred embodiment, a humanized antibody of the invention may comprise a light chain variable region comprising a CDR1 of amino acid sequence SEQ ID NO: 17 with zero to two amino acid substitutions, a CDR2 of amino acid sequence SEQ ID NO: 18 with zero to two amino acid substitutions, a CDR3 of amino acid sequence LSP, a heavy chain variable region comprising a CDR1 of amino acid sequence SEQ ID NO: 19 with zero to two amino acid substitutions, a CDR2 of amino acid sequence SEQ ID NO: 20 with zero to two amino acid substitutions, and a CDR3 of amino acid sequence SEQ ID NO: 21 with zero to two amino acid substitutions. In an exemplary embodiment, a humanized antibody of the invention may comprise a light chain variable region comprising a CDR1 of amino acid sequence SEQ ID NO: 17, a CDR2 of amino acid sequence SEQ ID NO: 18, a CDR3 of amino acid sequence LSP, a heavy chain variable region comprising a CDR1 of amino acid sequence SEQ ID NO: 19, a CDR2 of amino acid sequence SEQ ID NO: 20, and a CDR3 of amino acid sequence SEQ ID NO: 21. The invention also encompasses the corresponding nucleic acid sequences of SEQ ID NO:17, 18, 19, 20, and 21, which can readily be determined by one of skill in the art, and may be incorporated into a vector or other large DNA molecule, such as a chromosome, in order to express an antibody of the invention.

In another embodiment, a humanized antibody of the invention may comprise a light chain variable region comprising a CDR1 of amino acid sequence SEQ ID NO: 22 with zero to two amino acid substitutions, a CDR2 of amino acid sequence SEQ ID NO: 23 with zero to two amino acid substitutions, and a CDR3 of amino acid sequence SEQ ID NO: 24 with zero to two amino acid substitutions, or may comprise a heavy chain variable region comprising a CDR1 of amino acid sequence SEQ ID NO: 25 with zero to two amino acid substitutions, a CDR2 of amino acid sequence SEQ ID NO: 26 with zero to two amino acid substitutions, and a CDR3 of amino acid sequence SEQ ID NO: 27 with zero to two amino acid substitutions. In a preferred embodiment, a humanized antibody of the invention may comprise a light chain variable region comprising a CDR1 of amino acid sequence SEQ ID NO: 22 with zero to two amino acid substitutions, a CDR2 of amino acid sequence SEQ ID NO: 23 with zero to two amino acid substitutions, a CDR3 of amino acid sequence SEQ ID NO: 24 with zero to two amino acid substitutions, and a heavy chain variable region comprising a CDR1 of amino acid sequence SEQ ID NO: 25 with zero to two amino acid substitutions, a CDR2 of amino acid sequence SEQ ID NO: 26 with zero to two amino acid substitutions, and a CDR3 of amino acid sequence SEQ ID NO: 27 with zero to two amino acid substitutions. In an exemplary embodiment, a humanized antibody of the invention may comprise a light chain variable region comprising a CDR1 of amino acid sequence SEQ ID NO: 22, a CDR2 of amino acid sequence SEQ ID NO: 23, a CDR3 of amino acid sequence SEQ ID NO: 24, a heavy chain variable region comprising a CDR1 of amino acid sequence SEQ ID NO: 25, a CDR2 of amino acid sequence SEQ ID NO: 26, and a CDR3 of amino acid sequence SEQ ID NO: 27. The invention also encompasses the corresponding nucleic acid sequences of SEQ ID NO:22, 23, 24, 25, 26 and 27, which can readily be determined by one of skill in the art, and may be incorporated into a vector or other large DNA molecule, such as a chromosome, in order to express an antibody of the invention.

In still another embodiment, a humanized antibody of the invention may comprise a light chain variable region comprising a CDR1 of amino acid sequence of SEQ ID NO: 28 with zero to two amino acid substitutions, a CDR2 of amino acid sequence SEQ ID NO: 29 with zero to two amino acid substitutions, and a CDR3 of amino acid sequence SEQ ID NO: 30 with zero to two amino acid substitutions, or may comprise a heavy chain variable region comprising a CDR1 of amino acid sequence SEQ ID NO: 31 with zero to two amino acid substitutions, a CDR2 of amino acid sequence SEQ ID NO: 32 with zero to two amino acid substitutions, and a CDR3 of amino acid sequence SEQ ID NO: 33 with zero to two amino acid substitutions. In a preferred embodiment, a humanized antibody of the invention may comprise a light chain variable region comprising a CDR1 of amino acid sequence of SEQ ID NO: 28 with zero to two amino acid substitutions, a CDR2 of amino acid sequence SEQ ID NO: 29 with zero to two amino acid substitutions, a CDR3 of amino acid sequence SEQ ID NO: 30 with zero to two amino acid substitutions, and a heavy chain variable region comprising a CDR1 of amino acid sequence SEQ ID NO: 31 with zero to two amino acid substitutions, a CDR2 of amino acid sequence SEQ ID NO: 32 with zero to two amino acid substitutions, and a CDR3 of amino acid sequence SEQ ID NO: 33 with zero to two amino acid substitutions. In an exemplary embodiment, a humanized antibody of the invention may comprise a light chain variable region comprising a CDR1 of amino acid sequence of SEQ ID NO: 28, a CDR2 of amino acid sequence SEQ ID NO: 29, a CDR3 of amino acid sequence SEQ ID NO: 30, a heavy chain variable region comprising a CDR1 of amino acid sequence SEQ ID NO: 31, a CDR2 of amino acid sequence SEQ ID NO: 32, and a CDR3 of amino acid sequence SEQ ID NO: 33. The invention also encompasses the corresponding nucleic acid sequences of SEQ ID NO:28, 29, 30, 31, 32 and 33, which can readily be determined by one of skill in the art, and may be incorporated into a vector or other large DNA molecule, such as a chromosome, in order to express an antibody of the invention.

In yet another embodiment, a humanized antibody of the invention may comprise a light chain variable region comprising a CDR1 of amino acid sequence SEQ ID NO: 34 with zero to two amino acid substitutions, a CDR2 of amino acid sequence SEQ ID NO: 35 with zero to two amino acid substitutions, a CDR3 comprising amino acid sequence SEQ ID NO: 36 with zero to two amino acid substitutions, or may comprise a heavy chain variable region comprising a CDR1 comprising amino acid sequence SEQ ID NO: 37 with zero to two amino acid substitutions, a CDR2 comprising amino acid sequence SEQ ID NO: 38 with zero to two amino acid substitutions, and a CDR3 comprising amino acid sequence SEQ ID NO: 39 with zero to two amino acid substitutions. In a preferred embodiment, a humanized antibody of the invention may comprise a light chain variable region comprising a CDR1 comprising amino acid sequence SEQ ID NO: 34 with zero to two amino acid substitutions, a CDR2 of amino acid sequence SEQ ID NO: 35 with zero to two amino acid substitutions, a CDR3 comprising amino acid sequence SEQ ID NO: 36 with zero to two amino acid substitutions, a heavy chain variable region comprising a CDR1 comprising amino acid sequence SEQ ID NO: 37 with zero to two amino acid substitutions, a CDR2 comprising amino acid sequence SEQ ID NO: 38 with zero to two amino acid substitutions, and a CDR3 comprising amino acid sequence SEQ ID NO: 39 with zero to two amino acid substitutions. In an exemplary embodiment, a humanized antibody of the invention may comprise a light chain variable region comprising a CDR1 comprising amino acid sequence SEQ ID NO: 34, a CDR2 comprising amino acid sequence SEQ ID NO: 35, a CDR3 comprising amino acid sequence SEQ ID NO: 36, a heavy chain variable region comprising a CDR1 comprising amino acid sequence SEQ ID NO: 37, a CDR2 comprising amino acid sequence SEQ ID NO: 38, and a CDR3 comprising amino acid sequence SEQ ID NO: 39. The invention also encompasses the corresponding nucleic acid sequences of SEQ ID NO:34, 35, 36, 37, 38 and 39, which can readily be determined by one of skill in the art, and may be incorporated into a vector or other large DNA molecule, such as a chromosome, in order to express an antibody of the invention.

In an aspect, the antibodies in a pharmacologically effective amount preferred in pharmaceutical grade, including immunologically reactive fragments, are administered to a subject such as to a living subject to be treated for Aβ plaque associated symptoms. Administration is performed using standard effective techniques, include peripherally (i.e. not by administration into the central nervous system) or locally to the central nervous system. Peripheral administration includes but is not limited to intravenous, intraperitoneal, subcutaneous, pulmonary, transdermal, intramuscular, intranasal, buccal, sublingual, or suppository administration. Local administration, including directly into the central nervous system (CNS) includes but is not limited to via a lumbar, intraventricular or intraparenchymal catheter or using a surgically implanted controlled release formulation.

Pharmaceutical compositions for effective administration are deliberately designed to be appropriate for the selected mode of administration, and pharmaceutically acceptable excipients such as compatible dispersing agents, buffers, surfactants, preservatives, solubilizing agents, isotonicity agents, stabilizing agents and the like are used as appropriate. Remington's Pharmaceutical Sciences, Mack Publishing Co., Easton Pa., 16 Ed ISBN: 0-912734-04-3, latest edition, incorporated herein by reference in its entirety, provides a compendium of formulation techniques as are generally known to practitioners. It may be particularly useful to alter the solubility characteristics of the antibodies useful in this discovery, making them more lipophilic, for example, by encapsulating them in liposomes or by blocking polar groups.

Effective peripheral systemic delivery by intravenous or intraperitoneal or subcutaneous injection is a preferred method of administration to a living subject. Suitable vehicles for such injections are straightforward. In addition, however, administration may also be effected through the mucosal membranes by means of nasal aerosols or suppositories. Suitable formulations for such modes of administration are well known and typically include surfactants that facilitate cross-membrane transfer. Such surfactants are often derived from steroids or are cationic lipids, such as N-[1-(2,3-dioleoyl)propyl]-N,N,N-trimethyl ammonium chloride (DOTMA) or various compounds such as cholesterol hemisuccinate, phosphatidyl glycerols and the like.

The concentration of humanized antibody in formulations to be administered is an effective amount and ranges from as low as about 0.1% by weight to as much as about 15 or about 20% by weight and will be selected primarily based on fluid volumes, viscosities, and so forth, in accordance with the particular mode of administration selected if desired. A typical composition for injection to a living subject could be made up to contain 1 mL sterile buffered water of phosphate buffered saline and about 1-1000 mg of any one of or a combination of the humanized antibody of the present discovery. The formulation could be sterile filtered after making the formulation, or otherwise made microbiologically acceptable. A typical composition for intravenous infusion could have volumes between 1-250 mL of fluid, such as sterile Ringer's solution, and 1-100 mg per ml, or more in anti-ApoE antibody concentration. Therapeutic agents of the discovery can be frozen or lyophilized for storage and reconstituted in a suitable sterile carrier prior to use. Lyophilization and reconstitution may lead to varying degrees of antibody activity loss (e.g. with conventional immune globulins, IgM antibodies tend to have greater activity loss than IgG antibodies). Dosages administered are effective dosages and may have to be adjusted to compensate. The pH of the formulations generally pharmaceutical grade quality, will be selected to balance antibody stability (chemical and physical) and comfort to the subject when administered. Generally, a pH between 4 and 8 is tolerated. Doses will vary from individual to individual based on size, weight, and other physiobiological characteristics of the individual receiving the successful administration.

As used herein, the term “effective amount” means an amount of a substance such as a compound that leads to measurable and beneficial effects for the subject administered the substance, i.e., significant efficacy. The effective amount or dose of compound administered according to this discovery will be determined by the circumstances surrounding the case, including the compound administered, the route of administration, the status of the symptoms being treated and similar subject and administration situation considerations among other considerations. In an aspect, a typical dose contains from about 0.01 mg/kg to about 100 mg/kg of an anti-ApoE antibody described herein. Doses can range from about 0.05 mg/kg to about 50 mg/kg, more preferably from about 0.1 mg/kg to about 25 mg/kg. The frequency of dosing may be daily or once, twice, three times or more per week or per month, as needed as to effectively treat the symptoms.

The timing of administration of the treatment relative to the disease itself and duration of treatment will be determined by the circumstances surrounding the case. Treatment could begin immediately, such as at the site of the injury as administered by emergency medical personnel. Treatment could begin in a hospital or clinic itself, or at a later time after discharge from the hospital or after being seen in an outsubject clinic. Duration of treatment could range from a single dose administered on a one-time basis to a life-long course of therapeutic treatments.

Although the foregoing methods appear the most convenient and most appropriate and effective for administration of proteins such as humanized antibodies, by suitable adaptation, other effective techniques for administration, such as intraventricular administration, transdermal administration and oral administration may be employed provided proper formulation is utilized herein.

In addition, it may be desirable to employ controlled release formulations using biodegradable films and matrices, or osmotic mini-pumps, or delivery systems based on dextran beads, alginate, or collagen.

Typical dosage levels can be determined and optimized using standard clinical techniques and will be dependent on the mode of administration.

The following examples are included to demonstrate preferred embodiments of the invention. It should be appreciated by those of skill in the art that the techniques disclosed in the examples that follow represent techniques discovered by the inventors to function well in the practice of the invention. Those of skill in the art should, however, in light of the present disclosure, appreciate that many changes can be made in the specific embodiments that are disclosed and still obtain a like or similar result without departing from the spirit and scope of the invention, therefore all matter set forth or shown in the accompanying examples and drawings is to be interpreted as illustrative and not in a limiting sense.

### EXAMPLES

The following examples illustrate various iterations of the invention.

Antibodies specific for the ApoE protein were generated using hybridoma techniques, as described below in Examples 1 and 2. Specifically, four hybridomas were generated and ApoE antibodies were obtained from the hybridomas. The hybridomas HJ6.1, HJ6.2, HJ6.3, and HJ6.4 have been assigned ATCC Patent Deposit Designations PTA-11805, PTA-11806, PTA-11807, and PTA-11808 respectively.

### Example 1

### Preparation of Hybridomas

At first, 100 μl of the antigen peptide (ApoE, 1 mg/ml) in saline was mixed with an equal volume of Freund's complete adjuvant, emulsified and inoculated to the back of a mouse (Balb/c, 6 weeks of age). After 2 weeks, the mouse was re-immunized with a mixture of the 50 μl of the saline solution of antigen peptide (ApoE, 1 mg/ml) and Freund's incomplete adjuvant, emulsified by ultrasonic treatment, and after that, additional immunizations were carried out every week. On 40 days after immunization, the spleen was removed, the lymphocytes were harvested in PRM1640 medium (supplemented with penicillin and streptomycin) and treated with 0.17 M ammonium chloride to remove red blood cells. Isolated lymphocytes were fused with myeloma cells P3U1 strain derived from a mouse bone marrow tumor by the polyethylene glycol method (PEG4000) to obtain hybridoma cells. The hybridoma cells thus obtained were suspended in HAT medium with feeder cells and then distributed to 96-well plates and cultured for 15 days.

### Example 2

### Screening for the Monoclonal Antibody

The culture medium supernatants were recovered from wells in which the hybridoma cells obtained in Example 1 were cultured, and monoclonal antibodies which react with the antigen peptide by the enzyme-linked immunosorbent assay (ELISA) method were selected.

At first, 100 μl of 10 μg/ml antigen peptide was added to each well of 96-well plates, immobilized to the solid phase after keeping at 4° C. overnight and blocked with 200 μl of 10% calf serum at 37° C. overnight. Then, 100 μl of the culture medium supernatant of hybridoma cells was added to each well, reacted at 37° C. for 2 hours, and then horseradish peroxidase (HRP)-conjugated anti-mouse antibody, which was diluted 1000 folds, was added and reacted at 37° C. for 1 hour. The color was developed using Tetra Methyl Benzidine Microwell Peroxidase Substrate (TMB) as a substrate.

After terminating the reaction by adding 100 μl of 4N sulfuric acid, absorbance at 450 to 540 nm was measured, and the monoclonal antibodies, which showed the absorbance of about 3, were selected and cloned by the limited dilution method.

Mouse (Balb/c), injected with 0.5 ml pristine intraperitoneally 7 days and 3 days before, were inoculated intraperitoneally with selected monoclonal antibody-producing hybridoma cells, and ascites was collected about 10 days later. The collected ascites was kept at room temperature for 30 minutes, at 4° C. overnight and centrifuged at 15,000×rpm for 10 minutes, and then the supernatant was recovered.

The titers of selected monoclonal antibodies were measured by the ELISA method. ApoE was immobilized on a microwell plate, monoclonal antibodies were added, and after the reaction, color was developed using horse radish peroxidase (HRP)-conjugated anti-mouse antibody and TMB. The results indicate that selected monoclonal antibodies reacted in a concentration-dependent manner.

### Example 3

### Anti-ApoE Antibodies Cause Reduction in Plaque Load

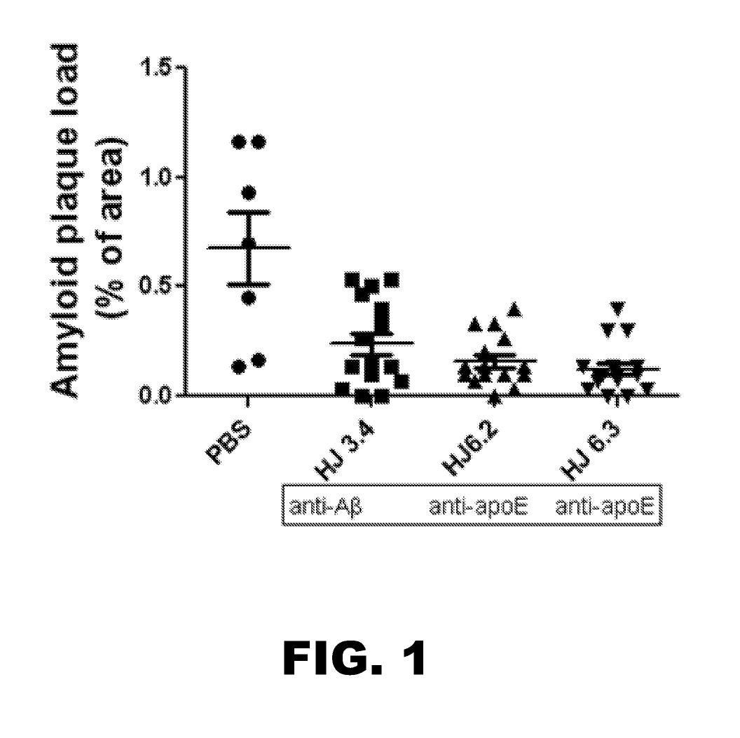

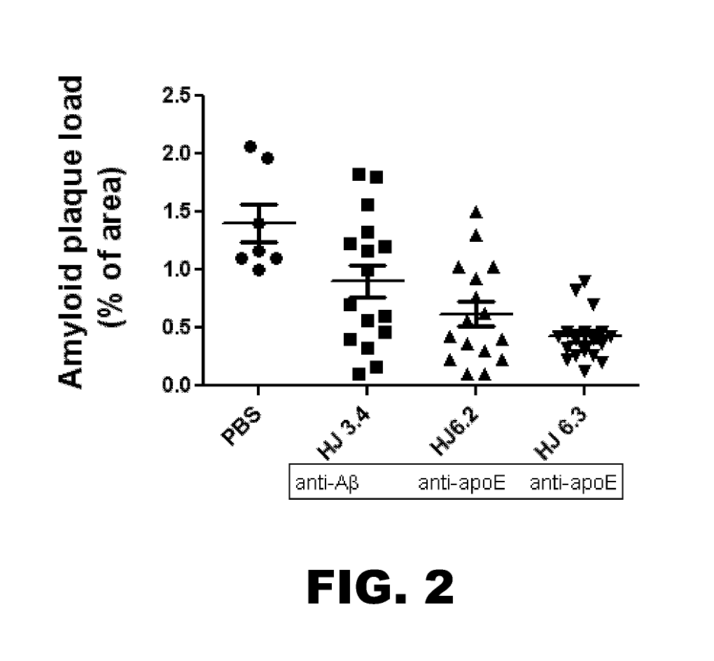

PS/APP mice, which exhibit age related amyloid plaque formation similar to that observed in Alzheimer's Disease subjects, are used as a mouse model for Alzheimer's Disease. PS/APP mice were treated intraperitoneally once weekly with 10 mg/kg with either an anti-Aβ antibody (HJ3.4) or one of two anti-ApoE antibodies (HJ6.2 or HJ6.3). Another group was treated with PBS as a control. Treatment was from 3-7 months of age. There was a strong effect of the anti-ApoE antibodies associated with a decrease in Aβ plaque load as demonstrated in both the hippocampus (FIG. 1) and cortex (FIG. 2).

### Example 4

### Binding of Anti-ApoE Antibodies to Human apoE

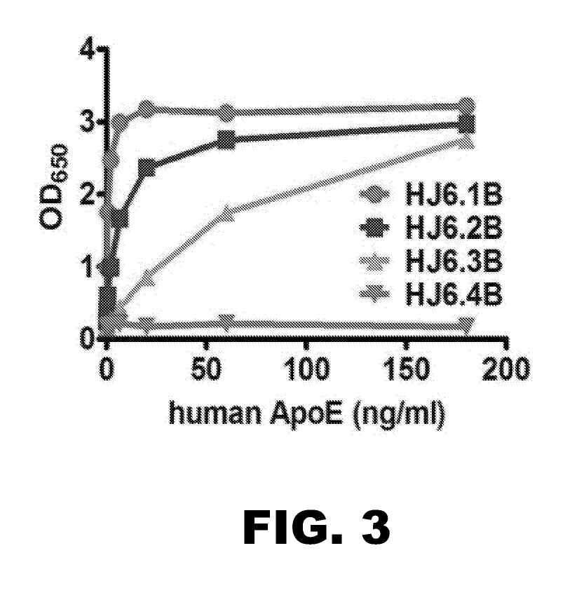

An ELISA sandwich assay was used to measure binding of anti-apoE HJ6.1, HJ6.2, HJ6.3, and HJ6.4 antibodies to human apoE. Monoclonal antibody WUE4 which binds to human apoE was first coated on an ELISA plate. Then differing concentrations of human apoE4 were applied. Biotinylated anti-apoE antibodies HJ6.1, HJ6.2, HJ6.3, and HJ6.4 were applied last and the optical density (OD) was read from a plate reader (FIG. 3).

These results show that anti-apoE antibodies HJ6.1, HJ6.2 and HJ6.3 bind to human apoE in an ELISA format with decreasing potency. However, anti-apoE antibody HJ6.4 does not bind to human apoE in this assay.

### Example 5

### Anti-apoE Antibodies Bind to Human apoE in Plasma but do not Affect Plasma Cholesterol

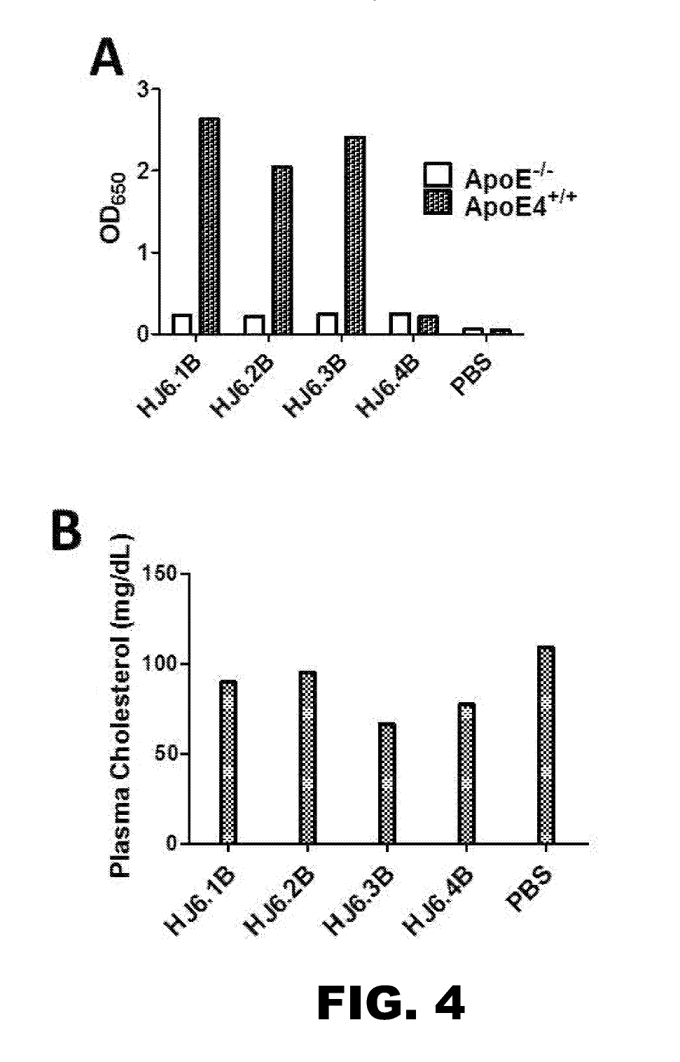

For these experiments, human ApoE4 knockin mice were used to measure plasma apoE/antibody complexes. ApoE4 knockin mice received 500 micrograms of biotinylated HJ6 mAbs intraperitoneally. Twenty fours later, the plasma was diluted (1:1000) and loaded to an ELISA plate coated with 5 μg/mL WUE4, which binds to human apoE. The ApoE/antibody complex was detected by a 1:5000 dilution HRP40. The optical density (OD) was read on a plate reader (FIG. 4A). These results show that HJ6.1, HJ6.2 and HJ6.3 antibodies bind to human apoE in plasma, whereas HJ6.4 did not bind to human apoE in plasma. No signal was obtained when this experiment was performed in apoE−/− mice.

Total plasma cholesterol was also measured in the same apoE4+/+ mice that received intraperitoneal injections of 500 micrograms of HJ6 series monoclonal antibodies HJ6.1, HJ6.2, HJ6.3, and HJ6.4. Plasma cholesterol did not change relative to animals that received phosphate buffered saline (FIG. 4B).

### Example 6

### HJ6 Series Antibodies to Human apoE Found in the Central Nervous System (CNS) after Periperhal Administration

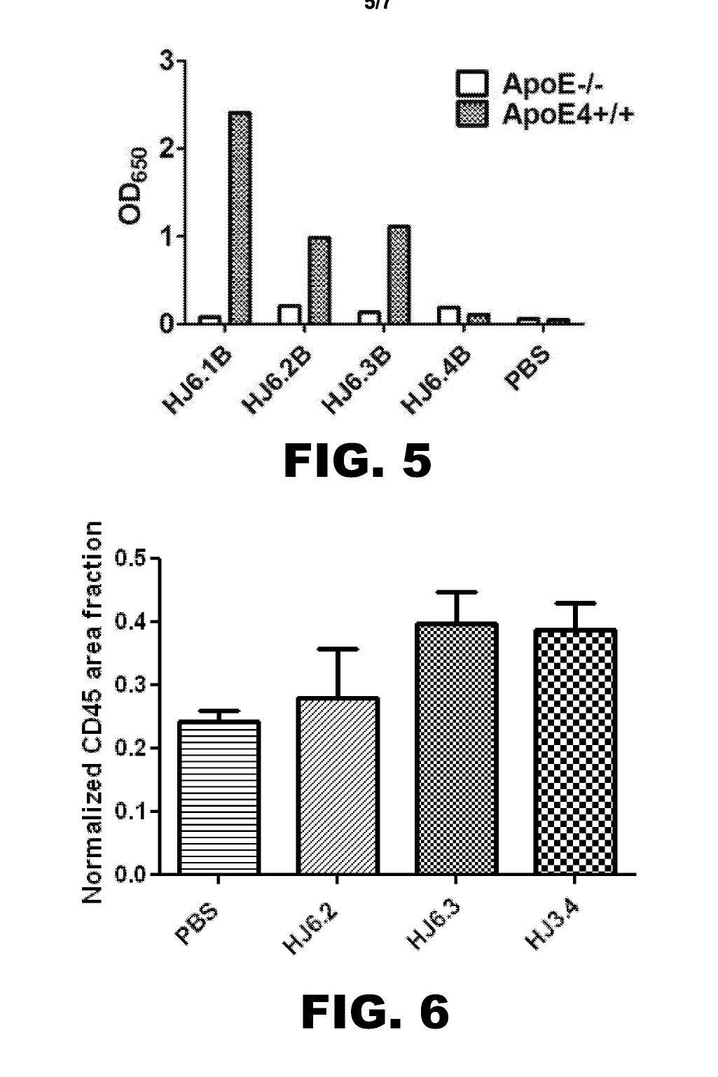

The cerebrospinal fluid (CSF) of ApoE4+/+ or ApoE4−/− mice that received HJ6 series mAbs or PBS in (see FIG. 4) was diluted (1:23) and the apoE4/biotinylated antibody complex in the CSF was captured by WUE4 and then detected by HRP40. The results demonstrate that in CSF there are complexes of apoE4 with the anti-apoE antibodies HJ6.1B, HJ6.2B or HJ6.3B (FIG. 5), suggesting that a fraction of the apoE mAbs were able to enter the CNS compartment and bind to apoE. The OD₆₅₀ followed the order HJ6.1B>HJ6.2B=HJ6.3B>>>HJ6.4B, which is consistent with the pattern seen for the ability of these antibodies to bind apoE on an ELISA plate (FIG. 3) and in plasma (FIG. 4).

### Example 7

### Microglial Activation in Mice Administered HJ6 Series Antibodies

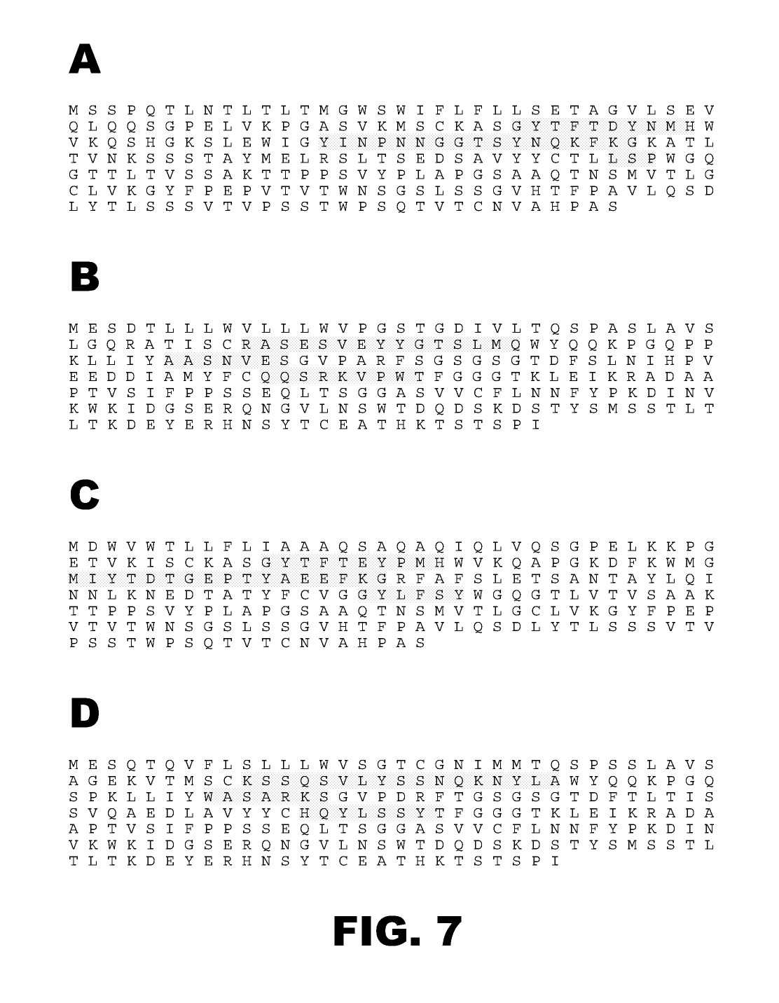

10 mg/kg of HJ6.2, HJ6.3 and HJ3.4 antibodies diluted in PBS, or the same volume of PBS was injected into PSAPP mice intraperitoneally at timepoint 0, day 3, day 6, and day 9. On day 10, brains were removed, fixed, and stained with antibody CD45 which marks activated microglia. There was an increase in the amount of cortex covered by activated microglia in the HJ6.3 and HJ3.4 injected mice. The PSAPP mice in this experiment express mouse apoE. These results show that short term administration of HJ6.3 (an anti-apoE antibody) and HJ3.4 (an anti-Aβ antibody) to 6 month old PSAPP mice that already have amyloid plaques over 10 days, results in microglial activation whereas one anti-apoE antibody (HJ6.2) does not appear to have this effect (FIG. 6).

What is claimed is:

1. A method of treating at least one symptom or sign of Aβ plaque associated symptoms in a subject which comprises administering an effective amount of at least one anti-ApoE antibody to that subject.

2. The method of claim 1, wherein the treating includes preventing, attenuating, reversing, or improving at least one symptom or sign of Aβ plaque associated symptoms in a subject.

3. The method of claim 1, wherein a symptom or sign of Aβ plaque associated symptoms includes impaired cognitive function, altered behavior, abnormal language function, emotional dysregulation, seizures, impaired nervous system structure or function, and an increased risk of development of Alzheimer's disease or cerebral amyloid angiopathy.

4. The method of claim 1, wherein the anti-ApoE antibody binds an epitope within the ApoE coding sequence.

5. The method of claim 1, wherein the administration comprises an effective systemic route of administration.

6. The method of claim 1, wherein the administration comprises an effective local route of administration including directly within the central nervous system.

7. The method of claim 1, wherein at least one anti-ApoE antibody comprises an amino acid sequence selected from the group consisting of SEQ ID NO:2, SEQ ID NO:4, SEQ ID NO:6, SEQ ID NO:8, SEQ ID NO:10, SEQ ID NO:12, SEQ ID NO:14, and SEQ ID NO:16.

8. The method of claim 1, wherein at least one anti-ApoE antibody is encoded by a nucleic acid sequence comprising a nucleic acid sequence selected from the group consisting of SEQ ID NO:1, SEQ ID NO:3, SEQ ID NO:5, SEQ ID NO:7, SEQ ID NO:9, SEQ ID NO:11, SEQ ID NO:13, and SEQ ID NO:15.

9. The method of claim 1, wherein at least one anti-ApoE antibody specifically binds ApoE and comprises a heavy chain CDR3 comprising the amino acid sequence of SEQ ID NO:21 with zero to two amino acid substitutions.

10. The method of claim 1, wherein at least one anti-ApoE antibody specifically binds ApoE and comprises a heavy chain CDR3 comprising the amino acid sequence of SEQ ID NO:27 with zero to two amino acid substitutions.

11. The method of claim 1, wherein at least one anti-ApoE antibody specifically binds ApoE and comprises a heavy chain CDR3 comprising the amino acid sequence of SEQ ID NO:33 with zero to two amino acid substitutions.

12. A method of decreasing the amyloid plaque load in the brain of a subject, the method comprising administering an effective amount of at least one anti-ApoE antibody to that subject.

13. The method of claim 12, wherein the anti-ApoE antibody binds an epitope within the ApoE coding sequence.

14. The method of claim 12, wherein the administration comprises an effective systemic route of administration.

15. The method of claim 12, wherein the administration comprises an effective local route of administration including directly within the central nervous system.

16. The method of claim 12, wherein the amyloid plaque loads are decreased in the hippocampus.

17. The method of claim 12, wherein the amyloid plaque loads are decreased in the cortex.

18. The method of claim 12, wherein at least one anti-ApoE antibody specifically binds ApoE and comprises a heavy chain CDR3 comprising the amino acid sequence of SEQ ID NO:21 with zero to two amino acid substitutions.

19. The method of claim 12, wherein at least one anti-ApoE antibody specifically binds ApoE and comprises a heavy chain CDR3 comprising the amino acid sequence of SEQ ID NO:27 with zero to two amino acid substitutions.

20. The method of claim 12, wherein at least one anti-ApoE antibody specifically binds ApoE and comprises a heavy chain CDR3 comprising the amino acid sequence of SEQ ID NO:33 with zero to two amino acid substitutions.


SEQUENCE LISTING (39 sequences)

SEQ ID NO: 1 - DNA, 687 bp, Artificial Sequence
  based on mus musculus
atggagtcagacacactcctgctatgggtgctgctgctctgggttccaggctccactggtgacattgtgctcacccaatctccagcttctttggctgtgtctctagggcagagagccaccatctcctgcagagccagtgaaagtgttgaatattatggcacaagtttaatgcagtggtaccaacagaaaccaggacagccacccaaactcctcatctatgctgcatccaacgtagaatctggggtccctgccaggtttagtggcagtgggtctgggacagacttcagcctcaacatccatcctgtggaggaggatgatattgcaatgtatttctgtcagcaaagtaggaaggttccgtggacgttcggtggaggcaccaagctggaaatcaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccatt

SEQ ID NO: 2 - DNA, 700 bp, Artificial Sequence
  based on mus musculus
atgtcctctcctcagacactgaacacactgactctaaccatgggatggagctggatctttctctttctcctgtcagaaactgcaggtgtcctctctgaggtccagctgcaacagtctggacctgagctggtgaagcctggggcttcagtgaagatgtcctgcaaggcttctggatacacattcactgactacaacatgcactgggtgaagcagagccatggaaagagccttgagtggattggatatattaaccctaacaatggtggtactagctacaaccagaagttcaagggcaaggccacattgactgtaaacaagtcctccagcacagcctacatggagctccgcagcctgacatcggaagattctgcagtctattactgtacacttctatccccgtggggccaaggcaccactctcacagtctcctcagccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagccagaccgtcacctgcaacgttgcccacccggccagca

SEQ ID NO: 3 - protein, 229 aa, Artificial Sequence
  based on mus musculus
MESDTLLLWVLLLWVPGSTGDIVLTQSPASLAVSLGQRATISCRASESVEYYGTSLMQWYQQKPGQPPKLLIYAASNVESGVPARFSGSGSGTDFSLNIHPVEEDDIAMYFCQQSRKVPWTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPI

SEQ ID NO: 4 - protein, 233 aa, Artificial Sequence
  based on mus musculus
MSSPQTLNTLTLTMGWSWIFLFLLSETAGVLSEVQLQQSGPELVKPGASVKMSCKASGYTFTDYNMHWVKQSHGKSLEWIGYINPNNGGTSYNQKFKGKATLTVNKSSSTAYMELRSLTSEDSAVYYCTLLSPWGQGTTLTVSSAKTTPPSVYPLAPGSAAQTNSMVTLGCLVKGYFPEPVTVTWNSGSLSSGVHTFPAVLQSDLYTLSSSVTVPSSTWPSQTVTCNVAHPAS

SEQ ID NO: 5 - DNA, 690 bp, Artificial Sequence
  based on mus musculus
atggaatcacagactcaggtcttcctctccctgctgctctgggtatctggtacctgtgggaacattatgatgacacagtcgccatcatctctggctgtgtctgcaggagaaaaggtcactatgagctgtaagtccagtcaaagtgttttatacagttcaaatcagaagaactacttggcctggtaccagcagaaaccagggcagtctcctaaactgctgatctactgggcatccgctaggaaatctggtgtccctgatcgcttcacaggcagtggatctgggacagattttactcttaccatcagcagtgtacaagctgaagacctggcagtttattactgtcatcaatacctctcctcgtacacgttcggaggggggaccaagctggaaataaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccatt

SEQ ID NO: 6 - DNA, 670 bp, Artificial Sequence
  based on mus musculus
atggattgggtgtggaccttgctattcctgatagcagctgcccaaagtgcccaagcacagatccagttggtgcagtctggacctgagctgaagaagcctggagagacagtcaagatctcctgcaaggcttctgggtataccttcacagaatatccaatgcactgggtgaagcaggctccaggaaaggatttcaagtggatgggcatgatatacaccgacactggagagccaacatatgctgaagagttcaagggacggtttgccttctctttggagacctctgccaacactgcctatttgcagatcaacaacctcaaaaatgaggacacggctacatatttctgtgtgggtggttacttgttttcttactggggccaagggactctggtcactgtctctgcagccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagccagaccgtcacctgcaacgttgcccacccggccagca

SEQ ID NO: 7 - protein, 230 aa, Artificial Sequence
  based on mus musculus
MESQTQVFLSLLLWVSGTCGNIMMTQSPSSLAVSAGEKVTMSCKSSQSVLYSSNQKNYLAWYQQKPGQSPKLLIYWASARKSGVPDRFTGSGSGTDFTLTISSVQAEDLAVYYCHQYLSSYTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPI

SEQ ID NO: 8 - protein, 223 aa, Artificial Sequence
  based on mus musculus
MDWVWTLLFLIAAAQSAQAQIQLVQSGPELKKPGETVKISCKASGYTFTEYPMHWVKQAPGKDFKWMGMIYTDTGEPTYAEEFKGRFAFSLETSANTAYLQINNLKNEDTATYFCVGGYLFSYWGQGTLVTVSAAKTTPPSVYPLAPGSAAQTNSMVTLGCLVKGYFPEPVTVTWNSGSLSSGVHTFPAVLQSDLYTLSSSVTVPSSTWPSQTVTCNVAHPAS

SEQ ID NO: 9 - DNA, 681 bp, Artificial Sequence
  based on mus musculus
atggacatgaggacccctgctcagtttcttggaatcttgttgctctggtttccaggtatcaaatgtgacatcaaactgacccagtctccatcttccatgtatgcatctctaggagagagagtcactatcacttgcaaggcgagtgaggacattaatagctatttaagctggttccagcagaaaccagggaaatctcctaagaccctgatctatcgtgcaaacagattggtagatggggtcccatcaaggttcagtggcagtggatctgggcaagattattctctcaccatcagcagcctggagtatgaagatatgggaatttattattgtctacagtatgatgagtttcctctcacgttcggtgctgggaccaagctggagctgacacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccatt

SEQ ID NO: 10 - DNA, 398 bp, Artificial Sequence
  based on mus musculus
atggcagcagctcaaagtatccaagcacagatccagttggtgcagtctggacctgagctgaagaagcctggagagacagtcaagatctcctgcaaggcttctgggtataccttcacaactgctggaatgcagtgggtgcaaaagatgccaggaaagggttttaagtggattggctggataaacacccactctggagagccaaaatatgcagaagatttcaagggacggtttgccttctctttggaaacctctgccagcactgcctatttacagataagcaacctcaaaaatgaggacacggctacgtatttctgtgcgagaatgggaggctatgctatggactactggggtcaaggaacctcagtcaccgtctcctctgccaaaacaacacccccatc

SEQ ID NO: 11 - protein, 227 aa, Artificial Sequence
  based on mus musculus
MDMRTPAQFLGILLLWFPGIKCDIKLTQSPSSMYASLGERVTITCKASEDINSYLSWFQQKPGKSPKTLIYRANRLVDGVPSRFSGSGSGQDYSLTISSLEYEDMGIYYCLQYDEFPLTFGAGTKLELTRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPI

SEQ ID NO: 12 - protein, 133 aa, Artificial Sequence
  based on mus musculus
MAAAQSIQAQIQLVQSGPELKKPGETVKISCKASGYTFTTAGMQWVQKMPGKGFKWIGWINTHSGEPKYAEDFKGRFAFSLETSASTAYLQISNLKNEDTATYFCARMGGYAMDYWGQGTSVTVSSAKTTPPS

SEQ ID NO: 13 - DNA, 690 bp, Artificial Sequence
  based on mus musculus
atggaatcacagactcaggtcttcctctccctgctgctctgggtatctggtacctgtgggaacattatgatgacacagtcgccatcatctctggctgtgtctgcaggagaaaaggtcactatgagctgtaagtccagtcaaagtgttttatacagttcaaatcagaagaactacttggcctggtaccagcagaaaccagggcagtctcctaaactgctgatctactgggcatccactagggaatctggtgtccctgatcgcttcacaggcagtggatctgggacagattttactcttaccatcaccaatgtacaagctgaagacctggcagtttattactgtcatcaatacctctcctcgtacacgttcggaggggggaccaagttggaaataaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccatt

SEQ ID NO: 14 - DNA, 670 bp, Artificial Sequence
  based on mus musculus
atggattgggtgtggaccttgctattcctgatagcagctgcccaaagtgcccaagcacagatccagttggtgcagtctggacctgagctgaagaagcctggagagacagtcaagatctcctgcaaggcttctgggtataccttcacagaatatccaatgcactgggtgaagcaggctccaggaaagggtttcaagtggatgggcatgatatacaccgacactggagagccaacatatgttgaagagttcaagggacggtttgccttctctttggagacctctgccagcactgcctatttgcagatcaacaacctcaaaaatgaggacacggctacatatttctgtgtaggtggttacttgttttcttactggggccaagggactctggtcactgtctctgcagccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagccagaccgtcacctgcaacgttgcccacccggccagca

SEQ ID NO: 15 - protein, 230 aa, Artificial Sequence
  based on mus musculus
MESQTQVFLSLLLWVSGTCGNIMMTQSPSSLAVSAGEKVTMSCKSSQSVLYSSNQKNYLAWYQQKPGQSPKLLIYWASTRESGVPDRFTGSGSGTDFTLTITNVQAEDLAVYYCHQYLSSYTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPI

SEQ ID NO: 16 - protein, 223 aa, Artificial Sequence
  based on mus musculus
MDWVWTLLFLIAAAQSAQAQIQLVQSGPELKKPGETVKISCKASGYTFTEYPMHWVKQAPGKGFKWMGMIYTDTGEPTYVEEFKGRFAFSLETSASTAYLQINNLKNEDTATYFCVGGYLFSYWGQGTLVTVSAAKTTPPSVYPLAPGSAAQTNSMVTLGCLVKGYFPEPVTVTWNSGSLSSGVHTFPAVLQSDLYTLSSSVTVPSSTWPSQTVTCNVAHPAS

SEQ ID NO: 17 - protein, 10 aa, Artificial Sequence
  based on mus musculus
GYTFTDYNMH

SEQ ID NO: 18 - protein, 17 aa, Artificial Sequence
  based on mus musculus
YINPNNGGTSYNQKFKG

SEQ ID NO: 19 - protein, 15 aa, Artificial Sequence
  based on mus musculus
RASESVEYYGTSLMQ

SEQ ID NO: 20 - protein, 7 aa, Artificial Sequence
  based on mus musculus
AASNVES

SEQ ID NO: 21 - protein, 9 aa, Artificial Sequence
  based on mus musculus
QQSRKVPWT

SEQ ID NO: 22 - protein, 10 aa, Artificial Sequence
  based on mus musculus
GYTFTEYPMH

SEQ ID NO: 23 - protein, 17 aa, Artificial Sequence
  based on mus musculus
MIYTDTGEPTYAEEFKG

SEQ ID NO: 24 - protein, 6 aa, Artificial Sequence
  based on mus musculus
GYLFSY

SEQ ID NO: 25 - protein, 17 aa, Artificial Sequence
  based on mus musculus
KSSQSVLYSSNQKNYLA

SEQ ID NO: 26 - protein, 7 aa, Artificial Sequence
  based on mus musculus
WASARKS

SEQ ID NO: 27 - protein, 8 aa, Artificial Sequence
  based on mus musculus
HQYLSSYT

SEQ ID NO: 28 - protein, 10 aa, Artificial Sequence
  based on mus musculus
GYTFTTAGMQ

SEQ ID NO: 29 - protein, 17 aa, Artificial Sequence
  based on mus musculus
WINTHSGEPKYAEDFKG

SEQ ID NO: 30 - protein, 8 aa, Artificial Sequence
  based on mus musculus
MGGYAMDY

SEQ ID NO: 31 - protein, 11 aa, Artificial Sequence
  based on mus musculus
KASEDINSYLS

SEQ ID NO: 32 - protein, 7 aa, Artificial Sequence
  based on mus musculus
RANRLVD

SEQ ID NO: 33 - protein, 9 aa, Artificial Sequence
  based on mus musculus
LQYDEFPLT

SEQ ID NO: 34 - protein, 10 aa, Artificial Sequence
  based on mus musculus
GYTFTEYPMH

SEQ ID NO: 35 - protein, 17 aa, Artificial Sequence
  based on mus musculus
MIYTDTGEPTYVEEFKG

SEQ ID NO: 36 - protein, 6 aa, Artificial Sequence
  based on mus musculus
GYLFSY

SEQ ID NO: 37 - protein, 17 aa, Artificial Sequence
  based on mus musculus
KSSQSVLYSSNQKNYLA

SEQ ID NO: 38 - protein, 7 aa, Artificial Sequence
  based on mus musculus
WASTRES

SEQ ID NO: 39 - protein, 8 aa, Artificial Sequence
  based on mus musculus
HQYLSSYT